(19) United States
(12) Patent Application Publication
(10) Pub. No.: US 2008/0161888 A1
(43) Pub. Date: Jul. 3, 2008

(54) Treatment of Skin by Spatial Modulation of Thermal Injury

(75) Inventor: James C. Hsia, Weston, MA (US)

Correspondence Address: PROSKAUER ROSE LLP, ONE INTERNATIONAL PLACE, BOSTON, MA, 02110, US

(73) Assignee: Candela Corporation, Wayland, MA (US)

(21) Appl. No.: 11/969,124

(22) Filed: Jan. 3, 2008

Related U.S. Application Data
(60) Provisional application No. 60/878,286, filed on Jan. 3, 2007.

(51) Int. Cl. A61B 18/04; A61B 18/02

(52) U.S. Cl. 607/100; 607/96

(57) ABSTRACT

A method for treating skin includes a first beam of radiation and a second beam of radiation. The first beam of radiation is delivered to a first region of skin to cause a plurality of sub-surface volumes of a first thermal injury, which can elicit a healing response from substantially undamaged skin adjacent to the sub-surface volumes of thermal injury. The second beam of radiation is delivered to a second region of skin to cause a second thermal injury, which can cause the skin to rejuvenate.

### CROSS-REFERENCE TO RELATED APPLICATION

This application claims the benefit of and priority to U.S. Provisional Application Ser. No. 60/878,286, filed Jan. 3, 2007, which is owned by the assignee of the instant application and the disclosure of which is incorporated herein by reference in its entirety.

### FIELD OF THE INVENTION

The invention relates generally to a skin treatment using radiation. The invention relates particularly to treating skin using a beam of radiation to cause spatially modulated thermal injury of the skin sufficient to elicit a healing response and improvement in the skin.

### BACKGROUND OF THE INVENTION

Ablative resurfacing of skin with lasers can be an effective treatment for skin conditions such as wrinkles. However, ablative resurfacing can have undesirable post-treatment side effects. For example, crusting, oozing, erythema can last up to 5 weeks. Furthermore, permanent scarring can be a possible long-term side effect of ablative resurfacing. Such side effects can be a deterrent to individuals who otherwise desire treatment.

Improved treatments with reduced side effects include forming sub-surface thermal damage of skin, while leaving the top layer intact by combining heating and surface cooling. However, the results of sub-surface methods can be less dramatic than those achieved by ablative resurfacing. Other improvements include fractional resurfacing techniques that treat skin in discrete spots and leave the skin between the spots untreated.

Fractional resurfacing technologies can have advantages including lower incidences of side-effects and expedited healing. These advantages can result from the undamaged regions providing blood and nutrients to the adjacent damaged regions and accelerating the healing process. Ablative resurfacing and technologies that include inducing uniform damage corresponding to coverage of an entire region can include higher efficacy at the cost of increased side effects.

### SUMMARY OF THE INVENTION

Skin can be treated by delivering a first beam of radiation to a first region of skin to cause a plurality of sub-surface volumes of a first thermal injury, which can elicit a healing response from substantially undamaged skin adjacent to the sub-surface volumes of thermal injury. A second beam of radiation can be delivered to a second region of skin to cause a second thermal injury, which can cause the skin to rejuvenate (e.g., mitigate wrinkles, age spots, and/or other characteristics associated with aged skin). Radiation can include any electromagnetic radiation such as ultraviolet, visible light, infrared, RF, and microwave. An advantage of the invention can be that a large target region is formed with damaged regions (e.g., thermal injuries) adjacent to substantially undamaged regions within the target region. The substantially undamaged regions can be less damaged or undamaged (e.g., less, substantially uninjured, or uninjured) than the damaged regions. In general, thermal injury can be injury to tissue due to heat from radiation exposure, including partial denaturation of collagen. Other advantages include: improved treatment efficacy initiating from first and/or second thermal injuries, high coverage of a treatment region corresponding to first and second thermal injuries, improved post-treatment healing initiating from substantially undamaged skin adjacent to first and/or second thermal injuries, and apparatus and methods for efficient and effective treatment of skin. In various embodiments, wounds in the skin that require long recovery periods are avoided. For example, effective treatment of skin can be provided without forming large or contiguous areas of acute injury or necrosis.

In one embodiment, the treatment can be used to treat wrinkles or for skin rejuvenation. However, the treatment is not limited to treating wrinkles or skin rejuvenation. A beam of radiation can be delivered non-invasively to affect the skin. In certain embodiments, the invention can be combined with other techniques known in the art to treat skin.

In one aspect, the invention features a method for treating skin including selecting a target region of skin defining a first region and a second region. A first beam of radiation is delivered to the first region of skin to cause a plurality of sub-surface volumes of a first thermal injury, which elicit a healing response from substantially undamaged skin adjacent to the sub-surface volumes of thermal injury. A second beam of radiation is delivered to the second region of skin to cause a second thermal injury, causing the skin to rejuvenate.

In another aspect, the invention features a method for treating skin including selecting a target region of skin defining a first region and a second region. A first region of skin is cooled to a first extent, and a second region of skin is cooled to a second extent. A first beam of radiation is delivered to the first region of skin to cause a plurality of sub-surface volumes of a first thermal injury, which elicit a healing response from substantially undamaged skin adjacent to the sub-surface volumes of thermal injury. A second beam of radiation is delivered to the second region of skin to cause a second thermal injury, causing the skin to rejuvenate.

In yet another aspect, the invention features an apparatus for treating skin including a source of a first beam of radiation, a source of a second beam of radiation, a modulator, and a radiation delivery device. The modulator receives the beams of radiation and modulates the first beam of radiation to target a first region of skin and the second beam of radiation to target a second region of skin. The radiation delivery device delivers the first beam of radiation to the first region of skin to cause a plurality of sub-surface volumes of a first thermal injury, which elicit a healing response from substantially undamaged skin adjacent to the sub-surface volumes of thermal injury. A second beam of radiation is delivered to the second region of skin to cause a second thermal injury, causing the skin to rejuvenate.

In still another aspect, the invention features an apparatus for treating skin including a source of a first beam of radiation, a source of a second beam of radiation, a cooling device, and a radiation delivery device. The cooling device cools a first region of the skin to a first extent and a second region of the skin to a second extent. The radiation delivery device delivers the first beam of radiation to the first region of skin cooled to the first extent to cause a plurality of sub-surface volumes of a first thermal injury, which elicit a healing response from substantially undamaged skin adjacent to the sub-surface volumes of thermal injury. A second beam of radiation is delivered to the second region of skin cooled to the second extent to cause a second thermal injury, causing the skin to rejuvenate.

In other examples, any of the aspects above, or any apparatus or method described herein, can include one or more of the following features.

In various embodiments, a sub-surface volume of the first thermal injury can include a volume of necrotic thermal injury. In some embodiments, collagen is partially denatured in the second region of skin, causing the skin to rejuvenate. In certain embodiments, collagen synthesis is accelerated in the second region of skin, causing the skin to rejuvenate. In one embodiment, the skin is rejuvenated by eliciting a healing response that produces substantially unwrinkled skin. The invention can include activating fibroblasts which can deposit increased amounts of extracellular matrix constituents in the second region of skin.

In various embodiments, the invention can include forming at least two thermal injuries including two first thermal injuries, two second thermal injuries, or one first thermal injury and one second thermal injury intervened by substantially undamaged skin. In some embodiments, the invention can include forming a plurality of noncontiguous second thermal injuries, disposed relative to the plurality of first thermal injuries, to form a pattern of interspersed first thermal injuries and second thermal injuries. In certain embodiments, the first region of skin is shallower than the second region of skin.

In various embodiments, the invention can include a method of (i) moving the first beam of radiation along a surface of the skin in a first pass to cause the first thermal injury and (ii) moving the second beam of radiation along the surface of the skin in a second pass to cause the second thermal injury. In some embodiments, the first beam of radiation and the second beam of radiation can differ in at least one parameter such as fluence, wavelength, and pulse duration.

In various embodiments, the invention can include a method of (i) cooling the first region of the skin to a first extent sufficient to induce the first thermal injury upon delivering the first beam of radiation to the first region and (ii) cooling the second region of the skin to a second extent sufficient to induce the second thermal injury upon delivering the second beam of radiation to the second region. In some embodiments, the first region is not cooled at all. The first and second beam of radiation can be the same.

Other aspects and advantages of the invention can become apparent from the following drawings and description, all of which illustrate the principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 shows an exemplary cross-section of skin.

FIG. 2 shows an exemplary system for treating skin.

FIG. 3A shows a cross-section of an exemplary region of skin treated according to an embodiment of the invention.

FIG. 3B shows a top view of the exemplary region of treated skin shown in FIG. 3A.

FIG. 4A shows a cross-section of an exemplary region of skin treated according to an embodiment of the invention.

FIG. 4B shows a top view of the exemplary region of treated skin shown in FIG. 4A.

FIG. 5A shows a relationship that can be used to control the temperature at a depth of a region of skin.

FIG. 5B shows a relationship that can be used to control a depth of thermal injury to a region of skin.

FIG. 6 shows an example of spatial modulation of a single beam of radiation.

FIG. 7 shows an example of spatial modulation of a plurality of beams of radiation.

FIG. 8 shows an exemplary embodiment of a device and method for treating skin.

FIG. 9 shows an exemplary embodiment of a device and method for treating skin that can be combined with the method shown in FIG. 8.

FIG. 10 shows an exemplary embodiment of treating skin in a single step.

FIG. 11 shows an exemplary treatment of skin using contact cooling.

FIG. 12 shows an exemplary treatment of skin using spray cooling.

### DESCRIPTION OF THE INVENTION

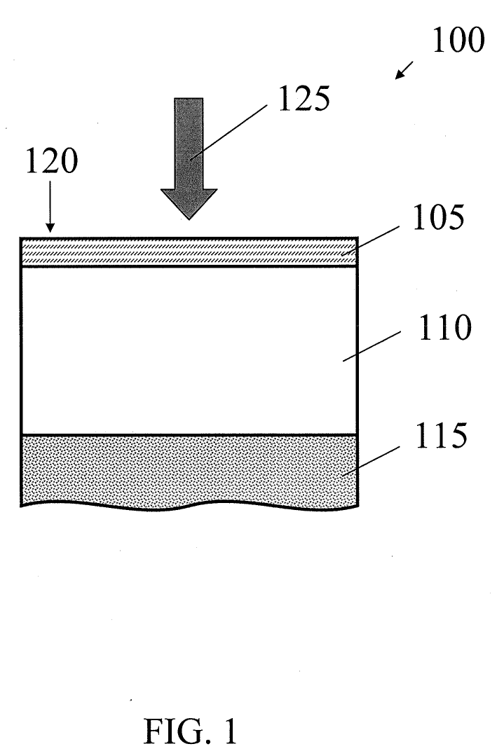

FIG. 1 shows an exemplary cross-section of skin 100 including a region of epidermis 105, a region of dermis 110, a region of subcutaneous tissue 115, and a surface of the skin 120. In one embodiment, the skin 100 can be a region of human skin with wrinkles. A beam of radiation 125 can be delivered to the skin 100 to treat at least a region of skin, including a region of epidermis 105 and/or a region of dermis 110. Skin treatments can include skin rejuvenation (e.g., mitigation of wrinkles, age spots, and/or other characteristics associated with aged skin) and treatments for wrinkles, vessels, pigmentation, age spots, scarring, and acne.

In general, a therapeutic injury can be induced with any electromagnetic radiation, including ultraviolet, visible light, infrared, RF, and microwave. In various embodiments, a therapeutic injury can be induced with electromagnetic radiation in the visible to infrared spectral region. A wavelength of light that penetrates into at least a portion of skin can be used. Chromophores can include blood (e.g., oxyhemoglobin and deoxyhemoglobin), collagen, melanin, fatty tissue, and water. Light sources can include lasers, light emitting diodes, or an incoherent source, and can be either pulsed or continuous. A light source can be coupled to a flexible optical fiber or light guide, which can be introduced proximally to a target region skin. The light source can operate at a wavelength with depth of penetration into skin that is less than the thickness of the target region of skin. In some embodiments, a therapeutic injury can be induced with RF energy and the energy source can include a RF generator. In one example, a RF delivery system can be capacitively coupled to the tissue to facilitate delivery of the RF energy. In certain embodiments, a therapeutic injury can be induced with microwave energy and the energy source can include a microwave generator. In one example, a microwave delivery system can target water in tissue.

In various embodiments, skin in a target region is heated to a critical temperature to cause thermal injury. In general, thermal injury can be injury to tissue due to heat resulting from radiation exposure. Thermal injury can include partial denaturation of collagen. For example, the healing response can be a remodeling of the skin's extracellular matrix. The healing response can include fibroblast-induced increases in extracellular matrix constituents such as collagen and glycosaminoglycans. In certain embodiments, the critical temperature is below about 100° C. In other embodiments, the critical temperature is below about 95° C., 90° C., 85° C., 80° C., 75° C., 70° C., 65° C., 60° C., 65° C., or 50° C. In some embodiments, the critical temperature is the temperature associated with ablation, coagulation, necrosis, and/or acute thermal injury of skin.

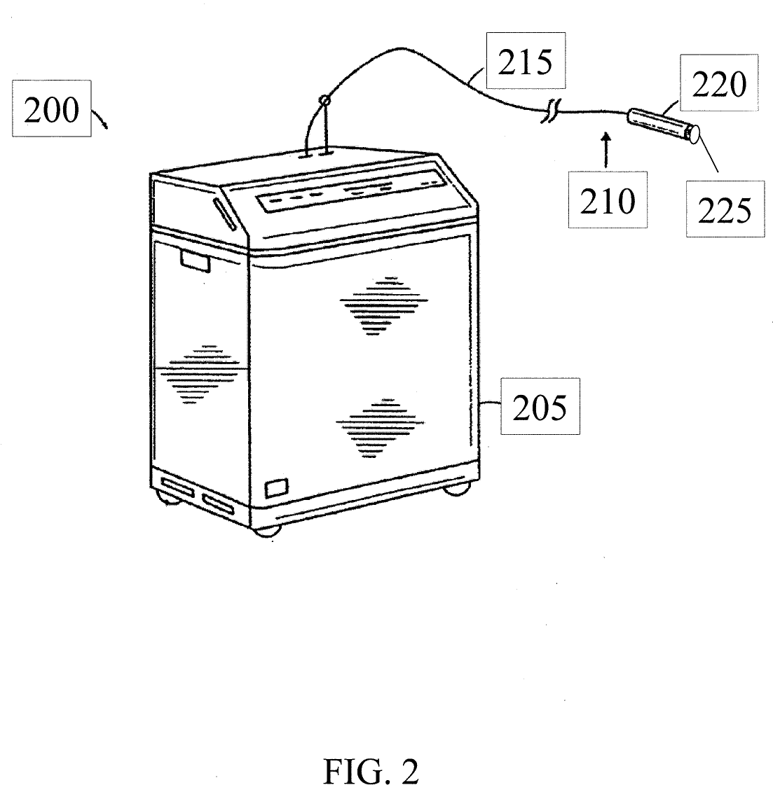

FIG. 2 shows an exemplary embodiment of a system 200 for treating skin. The system 200 can be used to non-invasively deliver a beam of radiation to a target region of skin. The system 200 includes an energy source 205 and a delivery system 210. In one embodiment, a beam of radiation provided by the energy source 205 is directed via the delivery system 210 to a target area. In the illustrated embodiment, the delivery system 210 includes a fiber 215 having a circular cross-section and a handpiece 220. A beam of radiation can be delivered by the fiber 215 to the handpiece 220, which can include an optical system (e.g., an optic or system of optics) to direct the beam of radiation to the target area. A user can hold or manipulate the handpiece 220 to irradiate the target area. The handpiece 220 can be positioned in contact with a skin surface, can be positioned adjacent a skin surface, can be positioned proximate a skin surface, can be positioned spaced from a skin surface, or a combination of the aforementioned. In the embodiment shown, the handpiece 220 includes a spacer 225 to space the delivery system 210 from the skin surface. In one embodiment, the spacer 225 can be a distance gauge, which can aid a practitioner with placement of the handpiece 220.

In various embodiments, the energy source 205 can be an incoherent light source, a coherent light source (e.g., a laser), a solid state laser, a diode laser, a fiber coupled diode laser array, an optically combined diode laser array, and/or a high power semiconductor laser. In some embodiments, a single source can provide both a first and a second beam of radiation. In certain embodiments, two or more sources can be used together to effect a treatment. For example, a first source can provide a first beam of radiation and a second source can provide a second beam of radiation. For example, an incoherent source can be used to provide a first beam of radiation while a coherent source provides a second beam of radiation. First and second beams of radiation can share a common wavelength or can have different wavelengths. In an embodiment using an incoherent light source or a coherent light source, a beam of radiation can be a pulsed beam, a scanned beam, or a gated continuous wave (CW) beam.

In various embodiments, the source of electromagnetic radiation can include a fluorescent pulsed light (FPL) or an intense pulsed light (IPL) system. However, the source of electromagnetic radiation can also include a laser, a diode, a coherent light source, an incoherent light source, or any other source of electromagnetic radiation. FPL technologies can utilize laser-dye impregnated polymer filters to convert unwanted energy from a xenon flashlamp into wavelengths that enhance the effectiveness of the intended applications. FPL technologies can be more energy efficient and can generate significantly less heat than comparative IPL systems. A FPL system can be adapted to operate as a multi-purpose treatment system by changing filters or handpieces to perform different procedures. For example, separate handpieces allow a practitioner to perform tattoo removal and other vascular treatments. An exemplary FPL system is described in U.S. Pat. No. 5,320,618, the disclosure of which is herein incorporated by reference in its entirety.

In various embodiments, a beam of radiation can have a wavelength between about 380 nm and about 2,600 nm. In certain embodiments, a beam of radiation can have a wavelength between about 1,200 nm and about 2,600 nm, between about 1,200 nm and about 1,800 nm, or between about 1,300 nm and about 1,600 nm. In one embodiment, a beam of radiation has a wavelength of about 1,500 nm. In other embodiments, a beam of radiation has a wavelength up to about 2,100 nm or up to about 2,200 nm.

In various embodiments, a beam of radiation can have a fluence of about 1 J/cm² to about 500 J/cm². For a given wavelength of radiation, a range of effective fluences can be approximated. Because radiation of wavelength between about 380 nm and about 2,600 nm is absorbed by water, and because skin is about 70% water, the absorption coefficient of skin can be approximated as 70% of the absorption coefficient of water. Because the absorption coefficient of water is a function of the wavelength of radiation, the desired fluence depends on the chosen wavelength of radiation. A fluence necessary to produce a desired damage depth can be approximated as a fluence that will raise the temperature to the critical temperature at the desired penetration depth, calculated as:

[3*μₐ*(μₐ+μₛ(1−g))]^{-0.5}

where μₐ, μₛ, and g are absorption coefficient, scattering coefficient, and the anisotropy factor of skin, respectively.

TABLE 1
Approximate fluence range to produce a first sub-zone
of damage using a given wavelength.

| | Fluence |
|---|---|
| Wavelength (nm) | (J/cm²) |
| 1180 | 100-498 |
| 1200 | 100-498 |
| 1220 | 109-545 |
| 1240 | 118-588 |
| 1260 | 116-582 |
| 1280 | 106-528 |
| 1300 | 93-466 |
| 1320 | 75-375 |
| 1340 | 65-326 |
| 1360 | 56-280 |
| 1380 | 29-146 |
| 1400 | 19-96 |
| 1420 | 14-70 |
| 1440 | 11-54 |
| 1460 | 11-55 |
| 1480 | 15-73 |
| 1500 | 18-88 |
| 1520 | 22-111 |
| 1540 | 20-101 |
| 1560 | 23-115 |
| 1580 | 26-130 |
| 1600 | 26-132 |
| 1620 | 31-153 |
| 1640 | 36-178 |
| 1660 | 39-196 |
| 1680 | 40-198 |
| 1700 | 40-200 |
| 1720 | 35-174 |
| 1740 | 29-145 |
| 1760 | 25-124 |
| 1780 | 26-128 |
| 1800 | 26-129 |
| 1820 | 23-115 |
| 1840 | 23-117 |
| 1860 | 22-109 |
| 1880 | 10-50 |
| 1900 | 5-23 |
| 1920 | 3-14 |
| 1940 | 3-13 |
| 1960 | 3-15 |
| 1980 | 3-17 |
| 2000 | 4-22 |
| 2020 | 6-28 |
| 2040 | 7-35 |
| 2060 | 8-40 |
| 2080 | 10-49 |
| 2100 | 12-58 |
| 2120 | 13-65 |
| 2140 | 15-76 |
| 2160 | 17-83 |
| 2180 | 18-90 |
| 2200 | 19-94 |
| 2220 | 19-96 |
| 2240 | 19-94 |
| 2260 | 18-90 |
| 2280 | 16-78 |
| 2300 | 14-69 |
| 2320 | 12-58 |
| 2340 | 10-49 |
| 2360 | 8-42 |
| 2380 | 7-36 |
| 2400 | 6-31 |
| 2420 | 5-26 |
| 2440 | 5-23 |
| 2460 | 4-20 |
| 2480 | 4-18 |
| 2500 | 3-17 |
| 2520 | 3-15 |
| 2540 | 3-14 |
| 2560 | 3-13 |
| 2580 | 2-12 |
| 2600 | 2-10 |

In various embodiments, a fluence used to produce a second sub-zone of damage is less than a fluence used to produce a first sub-zone of damage. In certain embodiments, a fluence used to produce a second sub-zone of damage is about 10% of a fluence used to produce a first sub-zone of damage.

In various embodiments, a desired penetration depth of light into the skin (and a corresponding depth of thermal injury) can be targeted by selecting a wavelength of a beam of radiation. For example, a water absorption coefficient can be taken from G. M. Hale and M. R. Querry, “Optical constants of water in the 200 nm to 200 μm wavelength region,” Appl. Opt., 12, 555-563, (1973) and an Optical Penetration Depth (OPD) can be calculated using a diffusion approximation. As described above, μₐ of skin is taken as μₐ of water multiplied by 0.7. The product of scattering coefficient and (1-anisotropy factor) is taken as 12 cm⁻¹.

TABLE 2
Approximate wavelength for a corresponding desired penetration depth.

| lambda | OPD | OPD |
|---|---|---|
| (nm) | (microns) | (mm) |
| 1180 | 1896.68 | 1.90 |
| 1200 | 1896.68 | 1.90 |
| 1220 | 1989.42 | 1.99 |
| 1240 | 2071.04 | 2.07 |
| 1260 | 2058.80 | 2.06 |
| 1280 | 1957.11 | 1.96 |
| 1300 | 1832.38 | 1.83 |
| 1320 | 1631.35 | 1.63 |
| 1340 | 1399.75 | 1.40 |
| 1360 | 1110.54 | 1.11 |
| 1380 | 692.68 | 0.69 |
| 1400 | 431.17 | 0.43 |
| 1420 | 279.87 | 0.28 |
| 1440 | 226.74 | 0.23 |
| 1460 | 229.34 | 0.23 |
| 1480 | 288.97 | 0.29 |
| 1500 | 333.69 | 0.33 |
| 1520 | 393.98 | 0.39 |
| 1540 | 445.51 | 0.45 |
| 1560 | 512.22 | 0.51 |
| 1580 | 583.96 | 0.58 |
| 1600 | 651.32 | 0.65 |
| 1620 | 713.46 | 0.71 |
| 1640 | 785.79 | 0.79 |
| 1660 | 831.29 | 0.83 |
| 1680 | 836.88 | 0.84 |
| 1700 | 842.55 | 0.84 |
| 1720 | 773.46 | 0.77 |
| 1740 | 689.83 | 0.69 |
| 1760 | 626.39 | 0.63 |
| 1780 | 575.87 | 0.58 |
| 1800 | 580.12 | 0.58 |
| 1820 | 538.51 | 0.54 |
| 1840 | 492.55 | 0.49 |
| 1860 | 391.16 | 0.39 |
| 1880 | 213.05 | 0.21 |
| 1900 | 111.13 | 0.11 |
| 1920 | 67.16 | 0.07 |
| 1940 | 64.38 | 0.06 |
| 1960 | 72.32 | 0.07 |
| 1980 | 82.28 | 0.08 |
| 2000 | 106.81 | 0.11 |
| 2020 | 128.89 | 0.13 |
| 2040 | 156.06 | 0.16 |
| 2060 | 176.11 | 0.18 |
| 2080 | 211.15 | 0.21 |
| 2100 | 239.41 | 0.24 |
| 2120 | 262.39 | 0.26 |
| 2140 | 296.35 | 0.30 |
| 2160 | 319.62 | 0.32 |
| 2180 | 338.03 | 0.34 |
| 2200 | 349.91 | 0.35 |
| 2220 | 356.02 | 0.36 |
| 2240 | 349.28 | 0.35 |
| 2260 | 338.77 | 0.34 |
| 2280 | 304.49 | 0.30 |
| 2300 | 277.13 | 0.28 |
| 2320 | 240.27 | 0.24 |
| 2340 | 208.26 | 0.21 |
| 2360 | 183.15 | 0.18 |
| 2380 | 161.22 | 0.16 |
| 2400 | 142.20 | 0.14 |
| 2420 | 121.74 | 0.12 |
| 2440 | 109.92 | 0.11 |
| 2460 | 97.31 | 0.10 |
| 2480 | 87.44 | 0.09 |
| 2500 | 83.58 | 0.08 |
| 2520 | 74.66 | 0.07 |
| 2540 | 70.44 | 0.07 |
| 2560 | 66.71 | 0.07 |
| 2580 | 58.99 | 0.06 |
| 2600 | 51.05 | 0.05 |

A skin treatment, utilizing the invention, can include selecting a target region of skin defining a first region and a second region. A first beam of radiation is delivered to the first region of skin to cause a plurality of sub-surface volumes of a first thermal injury, eliciting a healing response from substantially undamaged skin adjacent to the sub-surface volumes of thermal injury. A second beam of radiation is delivered to the second region of skin to cause a second thermal injury, causing the skin to rejuvenate. The first beam can be the same as the second beam.

A first thermal injury can be more severe than a second thermal injury. The plurality of first thermal injuries can include a volume of necrotic thermal injury. Forming the plurality of second thermal injuries can denature and/or partially denture collagen. Forming the plurality of second thermal injuries can also accelerate collagen synthesis. The plurality of second thermal injuries can elicit a healing response that produces substantially unwrinkled skin, and can cause the skin to rejuvenate. Forming the plurality of first and/or second thermal injuries can activate fibroblasts, which deposit increased amounts of extracellular matrix constituents in the second region of skin.

In various embodiments, the plurality of first thermal injuries can be substantially non-contiguous (e.g., intervened by substantially uninjured or less injured skin). In various embodiments, the plurality of second thermal injuries can be substantially non-contiguous. The first and second thermal injuries can be substantially non-contiguous. The first and second thermal injuries can be substantially sub-surface injuries and can leave the skin surface substantially uninjured.

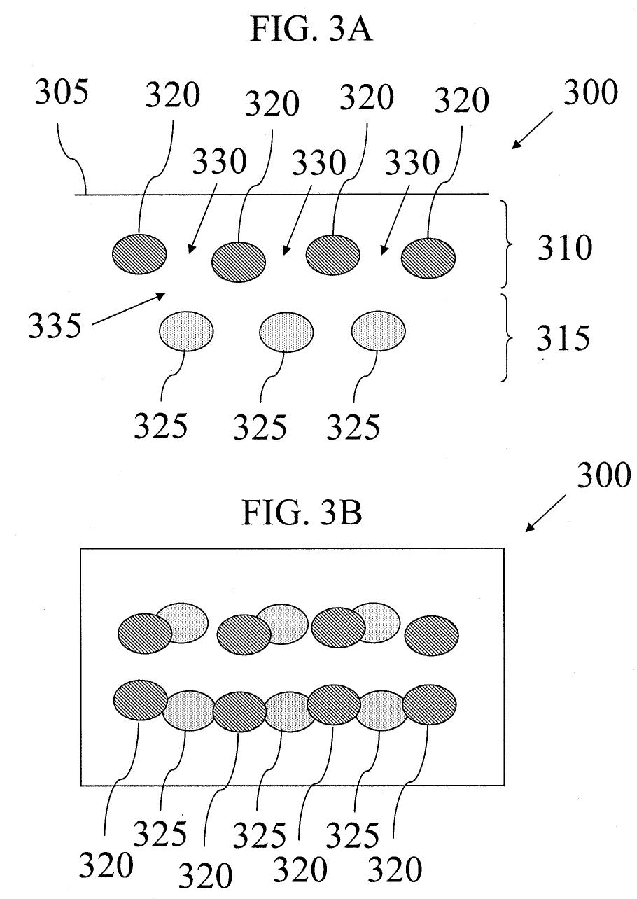

FIG. 3A shows a cross-section of an exemplary region of skin 300 including a skin surface 305, a first region 310 of skin at a first depth, a second region 315 of skin at a second depth, a plurality of first thermal injuries 320 in the first region 310, and a plurality of second thermal injuries 325 in the second region 315. Each plurality of thermal injuries can be separated by substantially undamaged skin 330. The thermal injuries at the first depth can be separated from the thermal injuries at the second depth by an intermediate region of substantially undamaged skin 335.

The first thermal injuries 320 can be more severe than the second thermal injuries 325. For example, the first thermal injuries 320 can be necrotic thermal injuries within the epidermis, and the second thermal injuries 325 can denature and/or partially denature collagen within the dermis. Necrotic thermal injuries elicit a healing response from the skin. Denaturing collagen can accelerate collagen synthesis, tighten skin, mitigate wrinkles, and/or elicit a healing response. An interspersed plurality of first thermal injuries 320 and second thermal injuries 325 can intensify the skin's healing response and accelerate recovery and healing, as compared to a large, continuous thermal injury. Healing can initiate from less injured or substantially undamaged skin 330 adjacent the plurality of first thermal injuries 320 and/or second thermal injuries 325.

FIG. 3B shows a top view of the region of skin 300 shown in FIG. 3A. The first and second thermal injuries can form less than about 100% coverage of a target region of skin, which can be measured as the area corresponding to the thermal injuries as seen from the skin surface. In some embodiments, the first and second thermal injuries can form about 100% coverage of a target region of skin.

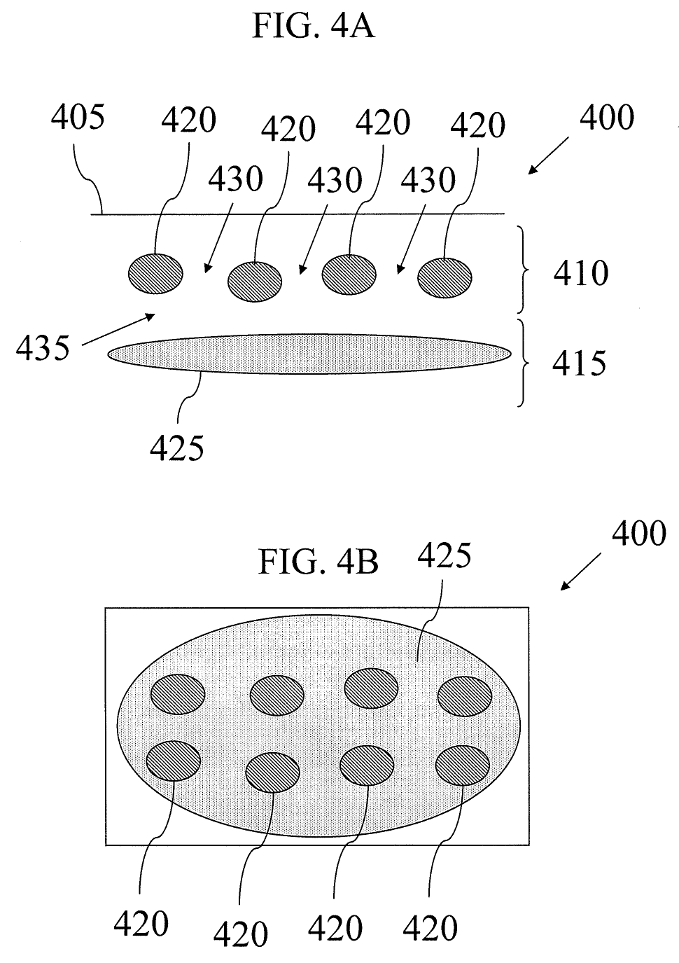

FIG. 4A shows a cross-section of an exemplary region of skin 400 including a skin surface 405, a first region 410 of skin at a first depth, a second region 415 of skin at a second depth, a plurality of first thermal injuries 420 in the first region 410, and a second thermal injury 425 in the second region 415. Each of the plurality of first thermal injuries 420 can be separated by substantially undamaged skin 430. The first thermal injuries 420 at the first depth can be separated from the second thermal injury 425 by an intermediate region of substantially undamaged skin 435.

The first thermal injuries 420 can be more severe than the second thermal injury 425. For example, the first thermal injuries 320 can be necrotic thermal injuries within the epidermis and the second thermal injury 425 can denature and/or partially denature collagen within the dermis. Necrotic thermal injuries elicit a healing response from the skin. Denaturing collagen can accelerate collagen synthesis, tighten skin, mitigate wrinkles, and/or elicit a healing response. The first thermal injuries 420 overlying a second thermal injury 425 can intensify the skin's healing response and accelerate recovery and healing, as compared to a large, continuous, severe thermal injury. Healing can initiate from less injured or substantially undamaged skin 430 adjacent the plurality of first thermal injuries 420 and/or second thermal injury 425.

FIG. 4B shows a top view of the region of skin 400 shown in FIG. 4A. The first and second thermal injuries can form about 100% coverage of a target region of skin, which can be measured as the area corresponding to the thermal injuries as seen from the skin surface. In some embodiments, the first and second thermal injuries can form less than about 100% coverage of a target region of skin.

As a beam or radiation penetrates skin, fluence (J/cm²) decreases in an approximately exponential fashion. The rate of decrease in fluence is dependent upon the absorption and scattering properties of skin. A local temperature increase due to absorbed radiation within the skin is a product of the local absorption coefficient and a local fluence divided by the volumetric specific heat. Since absorption and volumetric specific heat can be considered approximately constant within a region of skin, the local temperature rise can be considered proportional to fluence.

Thermal damage to skin forms at temperatures at, or exceeding, a critical temperature (T_{c}). Little or no thermal damage to skin forms at temperatures below T_{c}. Therefore, depth of thermal damage to skin is approximately equal to the depth of skin that is exposed to a temperature of, or exceeding, T_{c}.

In various embodiments, the first thermal injuries and/or the second thermal injuries are formed by heating the corresponding volume of skin to at least a critical temperature. In one embodiment, the temperatures forming the first thermal injuries and/or the second thermal injuries can vary at or above the critical temperature and, accordingly, the degree of thermal injury can also vary at or above a pre-determined amount. In another embodiment, the temperatures forming the first thermal injuries and/or the second thermal injuries can be substantially the same and, accordingly, the degree of thermal injury can also be substantially the same.

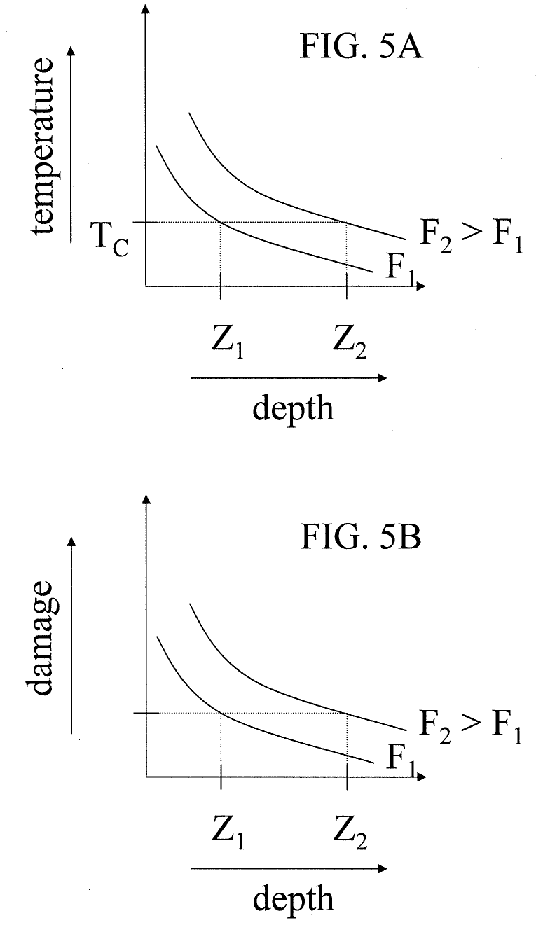

FIG. 5A shows a relationship that can be used to control the temperature at a depth of a region of skin. For a given temperature (T), a greater fluence can be selected if heating to a greater depth is desired and a lesser fluence can be selected if heating to a lesser depth is desired. This relationship can provide a method to control the depth to which a specific temperature is achieved.

FIG. 5B shows a relationship that can be used to control a depth of thermal injury to a region of skin. A lower fluence produces a smaller depth of thermal injury and a higher fluence produces a greater depth of thermal injury. For a given degree of damage, a greater fluence can be selected if a greater depth is desired and a lesser fluence can be selected if a lesser depth is desired. Therefore, by modulating fluence, one can modulate the depth and temperature within a region of skin.

To form deeper sub-zones of thermal injury, more penetrating wavelengths of radiation can be used. More penetrating wavelengths can be combined with longer pulse durations to increase thermal damage. In certain embodiments, more penetrating wavelengths can be combined with surface cooling to spare overlying tissue.

To form shallower sub-zones of thermal injury, less penetrating wavelengths can be used. Less penetrating wavelengths can be combined with shorter pulse durations. Less penetrating wavelengths can be used without surface cooling or with moderate cooling. Less penetrating wavelengths can also be used with surface cooling to maintain a temperature about a T_{c} (e.g., allow formation of thermal injury, but prevent necrosis or acute thermal injury).

In various embodiments a beam of radiation with varying intensity; a beam of radiation with varying exposure time or pulse duration; a beam of radiation with varying wavelengths wherein certain regions of a beam include a more penetrating wavelength and other parts include a less penetrating wavelength; and/or a beam of radiation with varying wavelengths with preferential absorption in different skin structures (e.g., a beam including wavelengths with strong water absorption interspersed with wavelengths with strong blood absorption) can be delivered to a target region of the skin to cause one or more thermal injuries.

Modulating pulse duration can modulate the depth and extent of a sub-zone of thermal injury. For a given fluence, longer pulse durations form a lesser extent of peak thermal injury than shorter pulse durations. To achieve the same extent of peak thermal injury, higher fluence can be used with longer pulse durations. Accordingly, longer pulse durations can result in a greater extent of thermal injury, for the same peak thermal injury, than shorter pulse duration. In various embodiments, a pulse duration can be between about 1 ms and about 1 min. Shorter pulse durations can be between about 1 ms and about 100 ms. Longer pulse durations can be between about 100 ms and about 1 min.

The first region of skin can be shallower than the second region of skin. A second thermal injury can be deeper than a first thermal injury. In some embodiments, a first thermal injury can be deeper that a second thermal injury. In one embodiment, first and second thermal injuries can be interspersed at varying depths. Depth can be measured from the skin surface. In various embodiments, a depth of a deeper thermal injury can be up to about 2 mm. In some embodiments, a depth of a deeper thermal injury can be about 400-800 μm. In certain embodiments, a depth of a shallower thermal injury can be up to about 200 μm. In some embodiments, a depth of a shallower thermal injury can be up to about 50 μm. In one embodiment, one or more deeper thermal injuries having a depth of about 400-800 μm are adjacent to one or more shallower thermal injuries having a depth of about 50 μm. In another embodiment, one or more deeper thermal injuries having a depth of about 400-800 μm are adjacent to one or more shallower thermal injuries having a depth of about 25 μm. In still another embodiment, one or more deeper thermal injuries having a depth of about 1.5 mm are adjacent to one or more shallower thermal injuries having a depth of about 50 μm.

In various embodiments, a diameter of a thermal damage can be between about 20 μm and about 2 mm. In some embodiments, a diameter of a thermal injury can be between about 100 μm and about 1000 μm. In various embodiments, spacing between thermal injuries can be between about 1 to about 10, or about 2 to about 5, times the diameter of the thermal injuries. Diameter of a thermal injury can be measured as the width of the projection of the thermal injury on the skin surface.

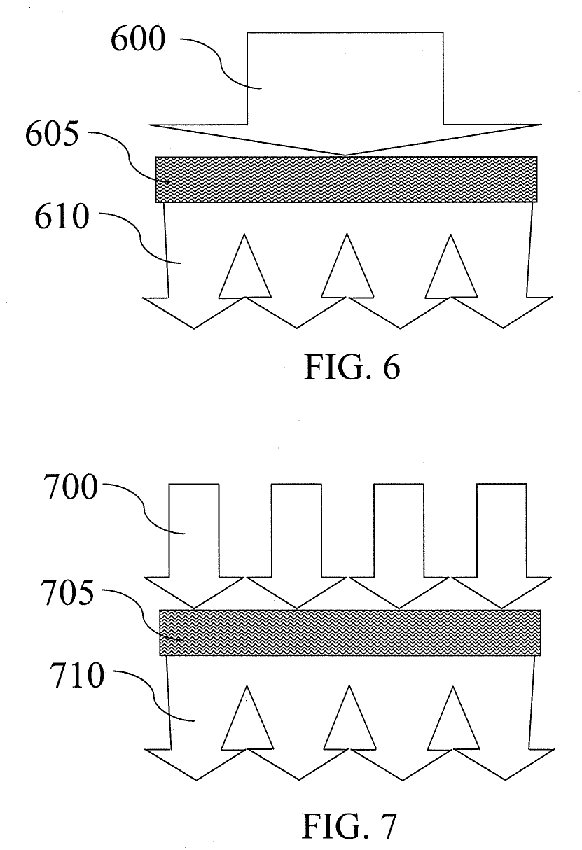

FIG. 6 shows an example of spatial modulation of a single beam of radiation. A single beam 600 of radiation is incident on a modulator 605, which forms a spatially modulated beam 610 of radiation. In various embodiments, the modulator 605 can be an optical device and/or an electromagnetic device. For example, the modulator 605 can be a lens, a micro lens array, a system of lenses, a diffractive optic in combination with a lens, or another optical device capable of varying the fluence of the single beam 600 of radiation. In certain embodiments, the modulator is an acousto-optic modulator. In certain embodiments, the single beam 600 can be modulated to form a plurality of discrete beams.

FIG. 7 shows another example of spatial modulation of a plurality of beams of radiation. A plurality of beams 700 of radiation are incident on a modulator 705, which forms the spatially modulated beam 710 of radiation. Regions of the discrete beams can overlap to form the spatially modulated beam of radiation 710.

In various embodiments, an optical fiber can be scanned over a surface of skin to deliver a spatially modulated beam of radiation to a target region of skin. In various embodiments an optical fiber bundle can be used to deliver a plurality of beams of radiation. In certain embodiments, the optical fiber bundle can operate in a scanning mode over a surface of skin. In certain embodiments, the optical fiber bundle can operate in a stamping mode over a surface of skin. The diameter of a region of a surface of skin treated in each stamp can range from about 1 mm to about 50 mm. An optical fiber can be a fiber laser.

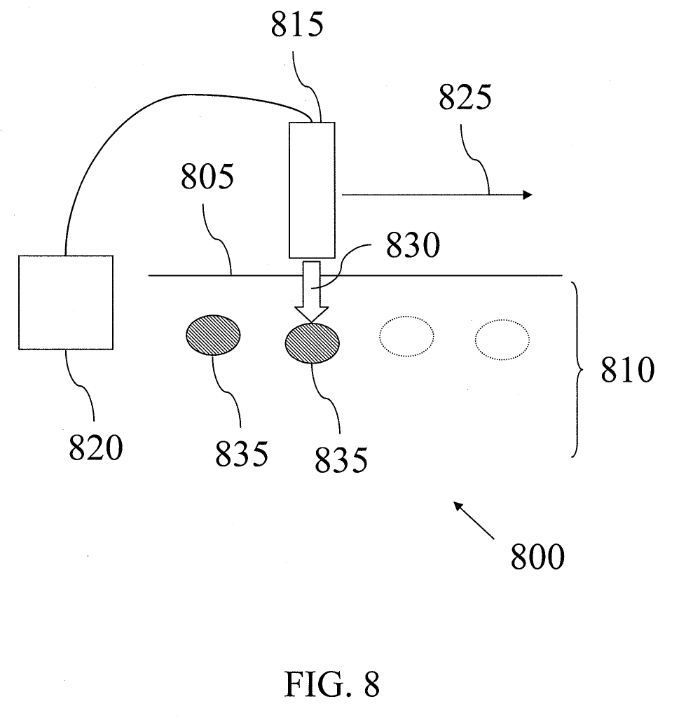

FIG. 8 shows an exemplary embodiment of a device and method for treating a region 800 of skin. The region 800 of skin includes a surface 805 and region of dermis and/or epidermis 810. A source 815 of a beam of radiation is coupled to a modulator 820. The source 815 can scan 825 along to the skin surface 805, to deliver a beam of radiation 830 to the skin. Scanning the source 815 and delivering the beam of radiation 830 can form one or more first thermal injuries 835. The modulator 820 can modulate the fluence, the intensity, or the wavelength of the beam 830 and/or the rate of translation of the source 815.

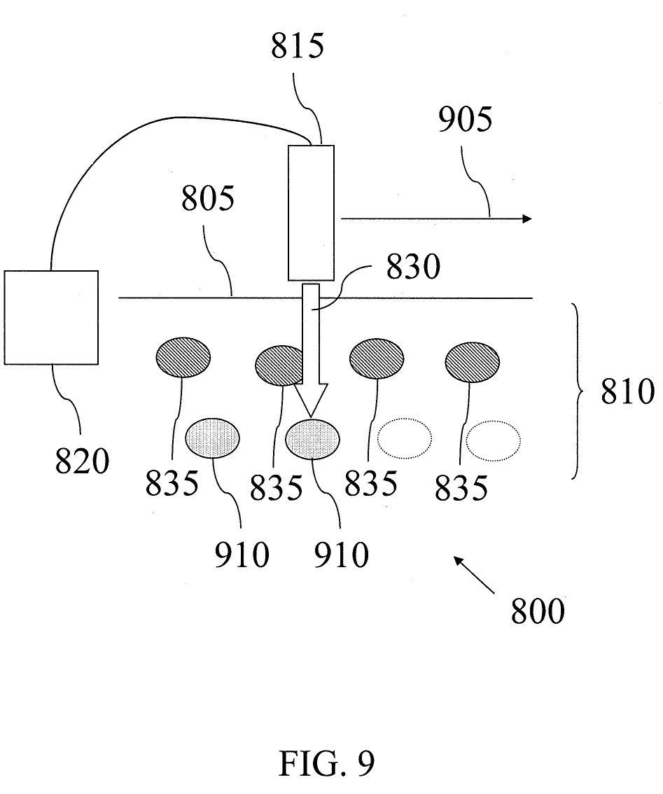

FIG. 9 shows an exemplary embodiment of a device and method for treating a region 800 of skin. In FIG. 8, the source 815 scanned 825 along to the skin surface 805, to deliver a beam of radiation 830 to the skin to form one or more first thermal injuries 835. In FIG. 9, the source 815 can make a separate and/or second scan 905 along to the skin surface 805, to deliver a beam of radiation 830 to the skin to form one or more second thermal injuries 910. The beams of radiation in the first pass (e.g., FIG. 8) and the second pass (e.g., FIG. 9) can differ in at least one parameter (e.g., fluence, wavelength, and/or pulse duration).

The first pass and the second pass can differ by parameters including cooling. For example, a first pass can employ extended cooling and radiation delivery. A second pass can employ rapid cooling and radiation delivery. The first pass will form deeper damage than the second pass.

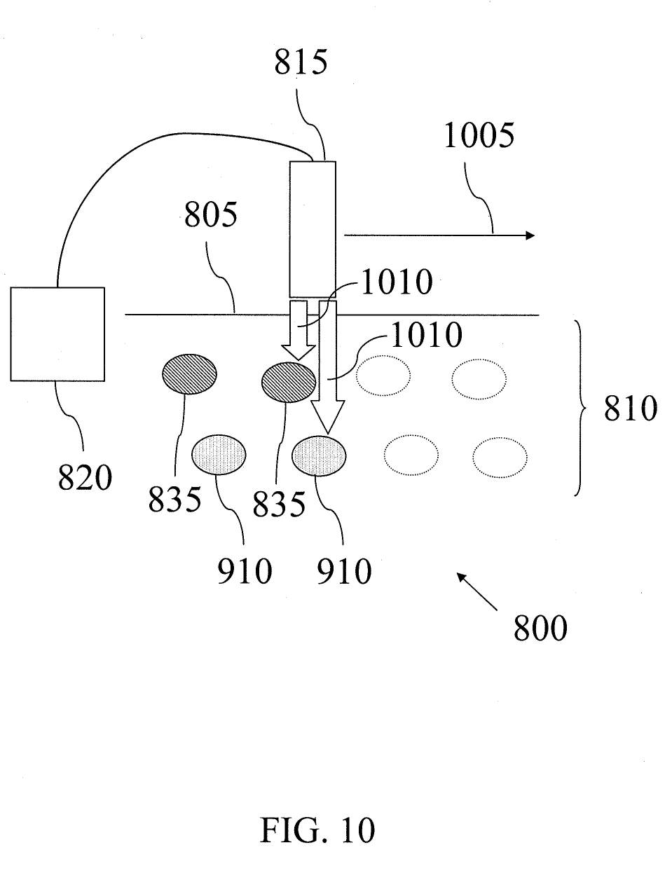

FIG. 10 shows an exemplary embodiment of a device and method for treating a region 800 of skin. In a single scan 1005, the source 815 translates along to the skin surface 805, to deliver a beam of radiation 1010 to the skin to form one or more first thermal injuries 835 and one or more second thermal injuries 910. The beam of radiation 1010 can include two or more beams of radiation and/or a modulated beam of radiation.

Varying the fluence, intensity, and/or wavelength of the beam 830 of radiation can vary the depth of thermal injury. For example, increasing the intensity of the beam 830 delivered to skin 800 can form a deeper zone of injury. Decreasing the intensity of the beam 830 delivered to skin 800 can form a shallow zone of injury. Varying the fluence, intensity, and/or wavelength of the beam 830 of radiation while scanning along to the skin surface 805 can form a spatially modulated thermal injury.

Varying the rate of translation of the beam 830 along the skin can vary the depth of thermal injury. Decreasing the rate over the skin surface 805 can increase the total fluence delivered, forming a more severe thermal injury. Increasing the rate over skin surface 805 can decrease the total fluence delivered, forming a less severe injury. Varying the rate of translation of the beam 830 of radiation while scanning along to the skin surface 805 can form a spatially modulated thermal injury.

In some embodiments, methods can include sequentially applying different combinations of radiation wavelength, intensity, and/or cooling such that a pattern of thermal injury achieved in a given pass is different than that achieved in a subsequent pass.

A cooling system can modulate the temperature in a region of skin and/or minimize unwanted thermal injury to untargeted skin. For example, the delivery system 200 shown in FIG. 2 can cool the skin before, during, or after delivery of radiation, or a combination of the aforementioned. Cooling can include applying a template to the skin and employing contact cooling, conduction cooling, evaporative spray cooling, convective air flow cooling, or a combination of the aforementioned. In one embodiment, the handpiece 220 includes a skin contacting portion that can contact a region of skin. The skin contacting portion can include a sapphire or glass window and a fluid passage containing a cooling fluid. The cooling fluid can be a fluorocarbon type cooling fluid, which can be transparent to the radiation used. The cooling fluid can circulate through the fluid passage and past the window to cool the skin.

A spray cooling device can use cryogen, water, or air as a coolant. In one embodiment, a dynamic cooling device (e.g., a DCD available from Candela Corporation) can cool the skin. For example, the delivery system 200 shown in FIG. 2 can include tubing for delivering a cooling fluid to the handpiece 220. The tubing can be connected to a container of a low boiling point fluid, and the handpiece can include a valve for delivering a spurt of the fluid to the skin. Heat can be extracted from the skin by evaporative cooling of the low boiling point fluid. In one embodiment, the fluid is a non-toxic substance with high vapor pressure at normal body temperature, such as a Freon or tetrafluoroethane.

By cooling only a region of the target region or by cooling different regions of the target region to different extents, the degree of thermal injury of regions of the target region can be controlled.

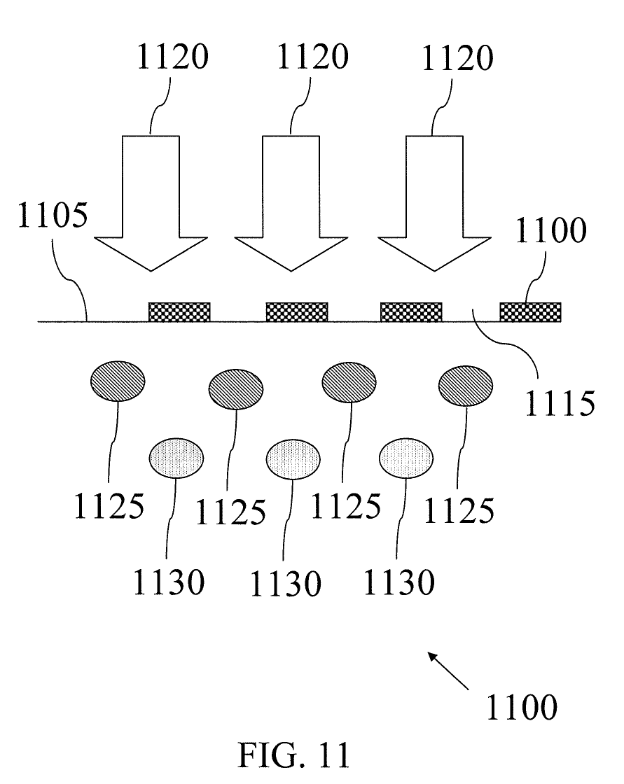

FIG. 11 shows an exemplary treatment of skin using cooling. A region of skin 1100 includes a skin surface 1105. A cooling plate 1110 is applied to the skin surface 1105 and a region 1115 of skin not in contact with the cooling plate 1110 is formed. A beam of radiation 1120 is delivered to the region of skin 1100 to treat the skin.

The cooling plate 1110 can cool different regions of the target region to different extents, thus modulating a spatial profile of temperature in the skin. For example, skin underlying the region 1115 not in contact with the cooling plate 1110 can be cooled to a first temperature, and skin underlying the cooling plate 1110 can be cooled to a second temperature. The first temperature is greater than the second temperature.

A region of skin 1100 with a spatially modulated temperature profile can create a spatially modulated pattern of thermal injury upon delivery of the beam of radiation 1120. For example, skin underlying the region 1115 not in contact with the cooling plate 1110 can correspond to one or more first thermal injuries 1125. Skin underlying the cooling plate 1110 can correspond to one or more second thermal injuries 1130. The second thermal injuries 1130 can be deeper than the first thermal injuries 1125 because the skin underlying the cooling plate 1110 is cooled by the cooling plate 1110 and therefore less affected by the beam of radiation 1120. The first thermal injuries 1125 can be more severe than the second thermal injuries 1130 because the skin underlying the region 1115 was not substantially cooled before delivery of the beam of radiation 1120.

In some embodiments, the cooling plate 1110 can be continuous (e.g., not have open regions), but have regions of varying thickness. Thicker regions of the cooling plate can extract more heat from the skin than thinner regions, thus a deeper zone of thermal injury can be formed under the thinner regions.

The cooling plate 1110 can be applied to the skin surface 1105 prior to or during delivery of the beam of radiation 1120. In some embodiments, the cooling plate 1110 can have at least one open region, corresponding to the region 1115 of skin not in contact with the cooling plate 1110.

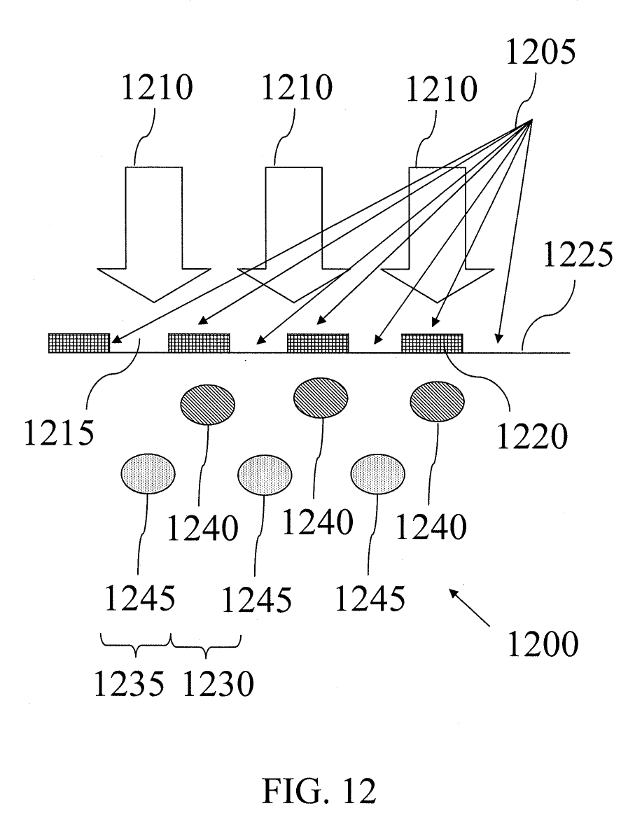

FIG. 12 shows another exemplary treatment of skin using cooling. A region of skin 1200 is treated with a cryogen spray 1205 and a beam 1210 of radiation. At least a region of the skin surface 1215 contacts a laser-transparent spray screen 1220, which can be applied to the skin surface 1225 before delivery of the cryogen spray 1205. The cryogen spray 1205 can be applied prior to or during delivery of the beam 1210 of radiation, to form a spatially modulated temperature profile in the skin 1200.

The screen 1220 can modulate a spatial profile of temperature in skin by preventing the cryogen spray 1205 from reaching at least a region of the skin surface 1225. In certain embodiments, the screen 1220 is not used and the cryogen spray 1205 is applied to the skin surface 1225 in pools of varying depth. A deeper pool can extract more heat from the skin surface 1225.

The beam 1210 of radiation is delivered to the surface 1225 of skin with at least one underlying region 1230 of skin of first temperature and at least one underlying region 1235 of skin of second temperature. The first temperature is greater than the second temperature. Delivery of the beam 1210 of radiation can increase the temperature in the target region. In a region 1230 of skin of first temperature, delivery of the beam 1210 of radiation can increase the temperature above a critical temperature, forming a first thermal injury 1240. In a region 1235 of skin of second temperature, delivery of the beam of radiation can increase the temperature to a second temperature below the first temperature. In a region 1235 of skin of second temperature, delivery of the beam 1210 of radiation can increase the temperature above a critical temperature, forming a second thermal injury 1245.

Cooling can be combined with control of fluence of the beam of radiation to control the depth of thermal injury. Cooling can also be combined with control of time of delivery of the beam of radiation to control the depth of thermal injury.

The time duration of cooling and of radiation application can be adjusted to maximize heating and thermal injury to the region proximate to the dermal interface. In tissue where the dermal interface is deeply situated, the cooling time can be lengthened such that cooling can be extended deeper into the skin. At the same time, the time duration of radiation application can be lengthened such that heat generated by the radiation in the region of dermis closer to the skin surface can be removed via thermal conduction and blood flow, thereby minimizing injury to the tissue overlying the dermal interface. Similarly, if the dermis overlying the dermal interface is thin, the time duration of cooling and of radiation application can be adjusted to be shorter, such that thermal injury is confined to the region proximate to the dermal interface.

Thermal injuries can assume different geometries. In various embodiments, thermal injuries can assume cylindrical, conical, cuboid, spheroid, ellipsoid, and/or ovoid geometries.

In another embodiment, a plurality of first thermal injuries adjacent to a plurality of second thermal injuries can form a one-dimensional array (e.g., a curvilinear pattern). Such a pattern can, for example, trace the contour of a wrinkle, vein, scar, or skin defect. In certain embodiments, methods can include varying a pattern over different parts of the skin to achieve different desired effects (e.g., to produce a pattern of surface injury in an area with surface wrinkles, while producing a pattern of sub-surface injury in an area for skin tightening with less surface injury).

While the invention has been particularly shown and described with reference to specific embodiments, it should be understood by those skilled in the art that various changes in form and detail may be made therein without departing from the spirit and scope of the invention as defined by the appended claims.

What is claimed is:

1. A method of treating skin comprising:
selecting a target region of skin defining a first region and a second region;
delivering a first beam of radiation to the first region of skin to cause a plurality of sub-surface volumes of a first thermal injury sufficient to elicit a healing response from substantially undamaged skin adjacent to the sub-surface volumes of thermal injury; and
delivering a second beam of radiation to the second region of skin to cause a second thermal injury sufficient to cause the skin to rejuvenate.

2. The method of claim 1 wherein each sub-surface volume of the first thermal injury comprises a volume of necrotic thermal injury.

3. The method of claim 1 further comprising partially denaturing collagen in the second region of skin to cause the skin to rejuvenate.

4. The method of claim 1 further comprising accelerating collagen synthesis in the second region of skin to cause the skin to rejuvenate.

5. The method of claim 1 wherein causing the skin to rejuvenate comprises eliciting a healing response that produces substantially unwrinkled skin.

6. The method of claim 1 further comprising activating fibroblasts which deposit increased amounts of extracellular matrix constituents in the second region of skin.

7. The method of claim 1 further comprising forming at least two thermal injuries comprising two first thermal injuries, two second thermal injuries, or one first thermal injury and one second thermal injury intervened by substantially undamaged skin.

8. The method of claim 1 further comprising forming a plurality of noncontiguous second thermal injuries, disposed relative to the plurality of first thermal injuries, to form a pattern of interspersed first thermal injuries and second thermal injuries.

9. The method of claim 1 wherein the first region of skin is shallower than the second region of skin.

10. The method of claim 1 further comprising:
moving the first beam of radiation along a surface of the skin in a first pass to cause the first thermal injury; and
moving the second beam of radiation along the surface of the skin in a second pass to cause the second thermal injury.

11. The method of claim 1 wherein the first beam of radiation and the second beam of radiation differ in at least one parameter.

12. The method of claim 11 wherein the parameter includes at least one of fluence, wavelength, and pulse duration.

13. The method of claim 1 wherein the first beam is the same as the second beam.

14. A method of treating skin comprising:
selecting a target region of skin defining a first region and a second region;
cooling the first region of skin to a first extent;
cooling the second region of skin to a second extent;
delivering a first beam of radiation to the first region of skin to cause a plurality of sub-surface volumes of a first thermal injury sufficient to elicit a healing response from substantially undamaged skin adjacent to the sub-surface volumes of thermal injury; and
delivering a second beam of radiation to the second region of skin to cause a second thermal injury sufficient to cause the skin to rejuvenate.

15. An apparatus for treating skin comprising:
a source of a first beam of radiation;
a source of a second beam of radiation;
a modulator receiving the first beam of radiation and the second beam of radiation and modulating the first beam of radiation and the second beam of radiation such that the first beam of radiation targets a first region of skin and the second beam of radiation targets a second region of skin; and
a device for delivering the first beam of radiation to the first region of skin to cause a plurality of sub-surface volumes of a first thermal injury sufficient to elicit a healing response initiating from substantially undamaged skin adjacent to the sub-surface volume of thermal injury, and for delivering the second beam of radiation to the second region of skin to cause a second thermal injury sufficient to cause the skin to rejuvenate.

16. An apparatus for treating skin comprising:
a source of a first beam of radiation;
a source of a second beam of radiation;
a cooling device adapted to cool a first region of the skin to a first extent and to cool a second region of the skin to a second extent; and
a device for delivering the first beam of radiation to the first region of skin cooled to the first extent to cause a plurality of sub-surface volumes of a first thermal injury sufficient to elicit a healing response initiating from substantially undamaged skin adjacent to the sub-surface volume of thermal injury, and for delivering the second beam of radiation to the second region of skin cooled to the second extent to cause a second thermal injury sufficient to cause the skin to rejuvenate.

17. The apparatus of claim 16 wherein the first beam of radiation and the second beam of radiation are the same.

18. The apparatus of claim 16 wherein the cooling device adapted to avoid cooling the first region of the skin.
